(19) United States
(12) Patent Application Publication
(10) Pub. No.: US 2016/0015669 A1
(43) Pub. Date: Jan. 21, 2016

(54) COMPOUND, 1, 3-DIOLEIN-2-LINOLEIN, AND PHARMACEUTICAL PREPARATION AND USE THEREOF

(71) Applicant: DAPENG LI, Hangzhou City (CN)

(72) Inventor: DAPENG LI, Hangzhou City (CN)

(73) Assignees: ZHEJIANG KANGLAITE GROUP CO., LTD., Hangzhou (CN); ZHEJIANG KANGLAITE PHARMACEUTICAL CO., LTD., Hangzhou (CN)

(21) Appl. No.: 14/732,791

(22) Filed: Jun. 8, 2015

(30) Foreign Application Priority Data
Jul. 18, 2014 (CN) 201410342454.7

(51) Int. Cl. A61K 31/231; C11B 3/10; B01D 15/42; A61K 9/48; B01D 15/08; B01D 15/22; C11B 1/10; A61K 9/00

(52) U.S. Cl. CPC A61K 31/231; C11B 1/104; C11B 3/10; A61K 9/0019; A61K 9/4833; A61K 9/4858; B01D 15/08; B01D 15/22; B01D 15/426

(57) ABSTRACT

The invention relates to a compound, 1,3-diolein-2-linolein, having a structure of formula (I), which is prepared by a process comprising the preparation of Coix seed oil from the Coix seed powder; the preliminary separation of 1,3-diolein-2-linolein; and the double-separation of 1,3-diolein-2-linolein. The invention also relates to pharmaceutical preparations of 1,3-diolein-2-linolein, and the use of this compound and pharmaceutical preparations thereof in the treatment of tumors.

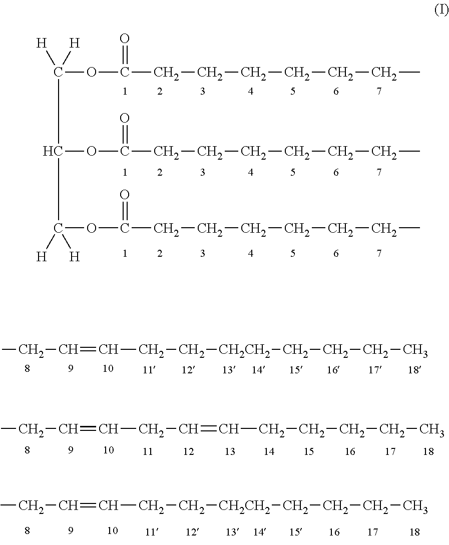

### CROSS REFERENCE TO RELATED PATENT APPLICATION

The present application claims a priority of the Chinese patent application CN201410342454.7 with filing date Jul. 18, 2014, which application is incorporated herein by reference.

### FIELD OF THE INVENTION

The present invention relates to fields of organic chemistry and pharmaceuticals, specifically, the present invention relates to a compound, 1,3-diolein-2-linolein, pharmaceutical preparations thereof, the process for the preparation of same and the use thereof in the treatment of tumors.

### BACKGROUND OF THE INVENTION

Coix seeds are dried ripe seeds of Coix lacryma-jobi L. var ma-yuen (Roman.), Stapf, a genus of plant in the Poaceae family. It is a dampness-eliminating drug and has been used as a medicinal and edible plant for a long time. Modern researches have found that Coix seeds have many pharmacological effects, such as analgesic anti-inflammatory, immunomodulatory, anti-ulcer, hypolipidemic and anti-obesity effects. In recent years, researchers, domestic and abroad, have studied the chemical composition of the Coix seed by using TLC, HPLC-MS, GC, etc., and found that the primary active ingredients in it includes esters, unsaturated fatty acids, saccharides and lactams. Among them, esters are the first discovered components having anti-tumor activities and the most reported chemical components attracting the most attention. The active ingredients of Kanglaite injection, which has been widely used in Chinese clinical applications, are esters extracted from Coix seed. It is shown in Yang Ling's research (Food Science, 2001, 22(5): 59-62) that the esters extracted from Coix seed contain triglycerides from 87.64% to 97.66% in mole percentage, and also monoglycerides, diglycerides and fatty acid esters. Further researches have indicated that triglycerides in Coix seed contain up to 8 ingredients in a relative content 3%, such as trilinolein (1), dilinolein-olein (2), palmitin-dilinolein (3), linolein-diolein (4) palmitin-linolein-olein (5), dipalmitin-linolein (6), triolein (7) and palmitin-diolein (8). The total contents of the 8 ingredients are over 90% of triglycerides. The complexity of the ingredients in Coix seed oil will inevitably be a great challenge which needs to be faced to in the quality control of the practical production process and in the safety of clinical applications.

Therefore, the development of a safe, effect and controllable medicine for the treatment of tumors became a focused issue of the invention. In this invention, one of the ingredients in triglycerides in Coix seed oil has been separated. A compound, 1,3-diolein-2-linolein, having a structure of formula (I) has been prepared for the first time. There is no report on the use of this compound and pharmaceutical preparations thereof in the treatment of tumors till now.

### SUMMARY OF THE INVENTION

The first aspect of the invention is to provide a compound, 1,3-diolein-2-linolein, having a structure of formula (I):

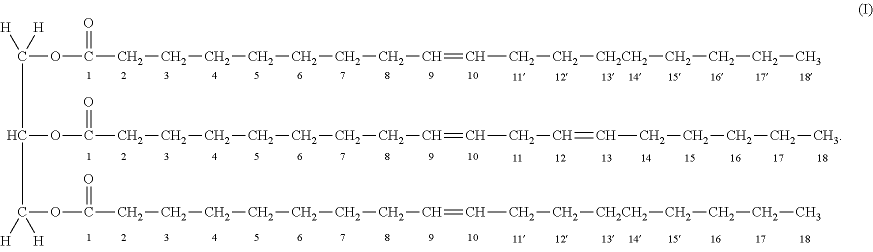

The compound of formula (I) is prepared by a process comprising steps of:
(1) preparation of Coix seed oil;
(2) preliminary separation of 1,3-diolein-2-linolein; and
(3) double-separation of 1,3-diolein-2-linolein.

The preparation of Coix seed oil in step (1) comprises steps of:

crushing Coix seeds having a moisture content ≦10% into 10-300 mesh powder and extracting said powder using a supercritical fluid extraction to afford a crude Coix seed oil; adding petroleum ether in an amount of 40-58% by weight of crude Coix seed oil and caustically refining this crude Coix seed oil with aqueous alkali, q.s.; after layering, adding neutral alumina and/or kaolin to the organic phase; filtering and sterilizing the filtrated oil via dry heat sterilization under vacuum at 150-180° C.; then cooling and filtering the sterilized oil to give Coix seed oil.

Preferably, the supercritical fluid extraction is the supercritical CO₂ extraction with an extraction temperature of 30-60° C., an extraction pressure of 19-24 Mpa, a separation temperature of 30-60° C., a separation pressure of 6-15 Mpa, a carbon dioxide flow rate of 10-5000 L/h, and an extraction time of 1-4 h;

the aqueous alkali can be a 0.5-3% KOH or NaOH aqueous solution; and

the step of adding neutral alumina and/or kaolin includes adding 3-8% activated neutral alumina by weight of crude Coix seed oil; filtering the mixture and heating the filtrate to 40-50° C.; adding 2-6% activated Kaolin by weight of crude Coix seed oil and filtering the mixture; removing solvent from the filtrate under vacuum; and adding 8-12% activated neutral alumina by weight of crude Coix seed oil, then filtering the mixture.

More preferably, the preparation of Coix seed oil in step (1) especially comprises steps of:

crushing Coix seeds having a moisture content ≦10% into 10-300 mesh powder and extracting the powder in a supercritical CO₂ extraction system wherein the CO₂ preheater, extractor and separation column are heated to reach an extraction temperature of 30-60° C. and a separation temperature of 30-60° C., then pressurized to reach an extraction pressure of 19-24 Mpa and a separation pressure of 6-15 Mpa; the CO₂, post the extraction of coix seed oil, enters into one or more resolution column(s), as needed, having a resolution temperature of 30-60 and a resolution pressure of 2-6 Mpa. The flow rate of CO₂ in the whole cycle is kept at 10-5000 L/h. The continuous extraction for 1-4 h affords a crude Coix seed oil from the lower end of the separation column; and

adding petroleum ether in an amount of 40-58% by weight of crude Coix seed oil to the crude Coix seed oil, and caustically refining the mixture with 2% NaOH solution, q.s.; after layering, collecting the organic phase and washing it with purified water; demulsifying the washed organic phase with acetone (q.s.); discarding the lower layer, adding activated neutral alumina in an amount of 3-8% by weight of crude Coix seed oil, and filtering the mixture; heating the filtrate up to 40-50 and adding activated kaolin in an amount of 2-6% by weight of crude Coix seed oil, and filtering the mixture; removing solvent from the filtrate under vacuum; and adding activated neutral alumina in an amount of 8-12% by weight of crude Coix seed oil, then filtering the mixture to give an oil; sterilizing the filtrated oil for 1-3 h via dry heat sterilization under vacuum at 150-180° C.; then cooling and filtering the sterilized oil through a ≦0.2 μm microporus membrane to give Coix seed oil.

The preliminary separation of 1,3-diolein-2-linolein in step (2) is especially as follows:

Instrument and method: using a preparative high performance liquid chromatograph in combination with an evaporative light-scattering detector, wherein the preparative chromatographic column is Agilent Zorbax SB-C18 (250 mm×21.2 mm, 7 μm); mobile phase A is acetonitrile and mobile phase B is tetrahydrofuran/acetonitrile (1:1); gradient conditions are mobile phase B: 0-27 min: 50%-60%, 27-35 min: 60-90%, 35-45 min: 90-100%; the flow rate is 18 mL/min; the injection volume is 200-3000; and the collection response is set at the peak level min=30 mV;

Preparation of sample: 0.1 g/ml Coix seed oil solution, prepared with mobile phase B; and

Collection of fraction: collecting the fraction of the chromatographic peak at the retention time of about 16 min and removing the solvent at a low temperature and a reducing pressure to obtain a preliminarily prepared sample.

The double-separation of 1,3-diolein-2-linolein in step (3) is especially as follows:

First separation: dissolving the sample obtained in the preliminary separation in n-hexane, q.s., with an ultrasonic wave to obtain a solution; injecting a sample (60 μl) of this solution in an analytical high performance liquid chromatograph (Column: Venusil XBP C18 (L) (10*250 mm, 5 μm, 150 Å)); eluting the sample with a mobile phase: acetonitrile/tetrahydrofuran (75:25) at a flow rate of 4 mL/min; detecting fractions with a DAD detector at 205 nm; collecting the fraction of chromatographic peak at the retention time of about 19 min and concentrating this fraction with nitrogen flow blowing; and

second separation: dissolving the sample obtained in the first separation in n-hexane, q.s., with an ultrasonic wave, to obtain a solution; injecting a sample (60 μl) of this solution in a high performance liquid chromatograph (column: Venusil CA (10*250 mm, 5 μm, 1000 Å)); eluting the sample with a mobile phase: n-hexane/ethanol (95:5) at a flow rate of 4 mL/min; detecting fractions with an ultraviolet detector at 205 nm; collecting the fraction of the chromatographic peak at retention time of about 2.5-5 min and concentrating the fraction with a nitrogen flow; and cryopreserving the final product at a low temperature.

The structure of the compound of the invention has been identified by its physiochemical properties, mass spectra, NMR, UV spectroscopy and IR spectroscopy.

1. Experimental Apparatus

HPLC: Agilent 1100 (DAD, ELSD), Agilent, USA;

NMR spectrometer: BRUKER AVANCE 500 MHz, Bruker, USA;

Mass spectrometer: Macromass GCT time of flight mass spectrometer (TOF), Waters, USA;

Finnigan TRACE DSQ-MS, Thermo Finnigan, USA;

HPLC-MS: Finnigan LCQ advantage MAX, Thermo Finnigan, USA;

IR spectrometer: Nicolet 6700, Thermo Finnigan, USA;

Polarimeter: Jasco P-1030, JASCO, Japan.

2. Experimental Methods

HPLC: 10 μl Solution of 20 mg pure product in 1 ml dichloromethane was determined in Agilent 1100 HPLC (Column: Agilent Zorbax SB-C18 (250 mm×4.6 mm, i.d. 5 μm); Mobile phase: dichloromethane/acetonitrile (35:65, v/v)).

LC-MS: The sample was determined in APCI positive ion mode in Finnigan LCQ advantage MAX in accordance of LC conditions.

IR: Pure product coated on the crystal surface of potassium bromide was determined in IR spectrometer (Nicolet 6700).

HR-EI and Qu.-EI: 5 μg pure product coated in the cell was determined in Macromass GCT time of flight mass spectrometer (TOF) in conditions of Source Temp 200° C., Sample Temp 400° C. and DIRECT 70 eV, and in Finnigan TRACE DSQ-MS in conditions of Source Temp 250° C., Sample Temp 250° C. and DIRECT 70 eV.

NMR: To 30-40 mg Pure product in a NMR tube was added 0.5 ml deuterated chloroform (CDCl₃) and injected highly pure argon. The NMR tube was closed meltingly in Argon shield with an alcohol blast burner, then ¹H-NMR, ¹³C-NMR, HSQC and HMBC were determined in a BRUKER 500M NMR spectrometer.

Specific rotation: Pure product, q.s., was dissolved in 1.50 ml chloroform, shaken up, and determined in JASCO P-1030.

3. Analysis of Spectrograms

The compound of formula (I) is a faint yellow oily liquid at room temperature.

HR-EI-MS: m/z=882.7678 (Calcd.=882.7676, C₅₇H₁₀₂O₆), Degree of unsaturation=7 (FIG. 1).

APCI-MS: 883.4, [M+H]⁺ signal, and fragment signals [M+H-linoleic acid]⁺=603.3 and [M+H-oleic acid]⁺=601.2 simultaneously appeared. It is deduced that there are two acyl-chains, oleic acid and linoleic acid (FIG. 2).

IR (KBr flim): 1747, 1163, 1097; 2925, 2855, 723; 3007, 1655 cm⁻¹ (FIG. 3).

¹H-NMR: δ5.27 (m, 1H), 4.30 (dd, J=4.29 Hz, J=11.9 Hz, 2H), 4.15 (dd, J=5.9 Hz, J=11.9 Hz, 2H), 2.33 (m, 6H), 2.77 (t, 2H), 5.36 (m, 8H), 0.88 (m, 9H) (FIG. 4).

¹³C-NMR: δ 14.10, 14.14, 22.60, 22.71, 24.86, 24.90, 25.65, 27.2, 27.22, 27.24, 29.07, 29.79, 31.55, 31.93, 34.05, 34.21, 62.12, 68.89, 127.90, 128.10, 129.73, 130.00, 130.03, 130.24, 172.87, 173.29 (FIG. 5).

The second aspect of the invention is to provide a pharmaceutical composition, comprising a therapeutically effective amount of at least one active ingredient including the compound of formula (I) and one or more pharmaceutically acceptable carriers.

Preferably, the pharmaceutical composition is a pharmaceutical preparation in a form of unit dose, comprising 0.1 mg-1000 mg of compound of formula (I) and 0.01%-99.99% pharmaceutically acceptable carriers by weight of the pharmaceutical preparation.

The pharmaceutical composition of the invention can be any form of medicine, such as common tables, sugar coating tables, film coating tables, enteric coating tables, capsules including hard capsules and soft capsules, oral liquids, buccal preparations, granules, electuaries, pills, powders, mastics, sublimed preparations, suspensions, pulvis, solutions, injections, suppositories, ointments, plasters, creams, sprays, drops and patches.

The pharmaceutically acceptable carriers are selected from pharmaceutically conventional dilutions, excipients, fillers, emulsifiers, binders, lubricants, absorption accelerators, surfactants, disintegrants, lubricants and antioxidants, if necessary, flavoring agents, sweeteners, preservative and/or coloring agents.

Especially, the pharmaceutically acceptable carriers are selected from one or more in the group consisting of: mannitol, sorbitol, sodium metabisulfite, sodium bisulfite, sodium thiosulfate, cysteine hydrochloride, thioglycolic acid, methionine, soybean lecithin, vitamin C, vitamin E, EDTA disodium, EDTA calcium sodium, a monovalent alkali metal carbonate, acetate, phosphate or its aqueous solution, hydrochloric acid, acetic acid, sulfuric acid, phosphoric acid, amino acids, sodium chloride, potassium chloride, sodium lacetate, ethylparaben solution, benzoic acid, potassium sorbate, chlorhexidine acetate, xylitol, maltose, glucose, fructose, dextran, glycine, starch, sucrose, lactose, mannitol, and silicic derivatives, cellulose and its derivatives, alginates, gelatin, polyvinyl pyrrolidone, glycerin, Tween 80, agar-agar, calcium carbonate, calcium bicarbonate, surfactant, polyethylene glycol, cyclodextrin, β-cyclodextrin, phospholipid material, kaolin, talc, and calcium stearate or magnesium stearate.

Preferably, the pharmaceutical preparation of the invention can be an oral solid preparation, an oral liquid preparation, or an injection.

Preferably, the oral solid preparation is selected from any one of capsules, tablets, dripping pills, granules and concentrated pills.

Preferably, the oral liquid preparation is selected from any one of aqueous or oily suspensions, solutions, emulsions, syrups or elixirs, and a dry product that can be reconstructed by water or other suitable carrier prior to use.

Preferably, the injection is selected from any one of injection, lyophilized powder and aqueous injection.

Preferably, the pharmaceutical preparation of the invention is an injection comprising the following components:

| | | |
|---|---|---|
| 1,3-diolein-2-linolein | 100-300 | g |
| Soybean lecithin for Injection or | 10-40 | g |
| soybean lecithin acceptable for injection | | |
| Glycerin for Injection or | 15-50 | g |
| glycerin acceptable for injection | | |
| Water for injection adds to | 1000 | mL. |

This injection is prepared by a method comprising steps of:

adding appropriate amount of water for injection to a formulated amount of soybean lecithin for injection or soybean lecithin acceptable for injection; dispersing the mixture with a high shear dispersing emulsifier to give a dispersion without bulk or granular solid; adding a formulated amount of glycerin for injection or glycerin acceptable for injection; then adding water for injection to a specified amount, and stirring the mixture to give a water phase;

weighing a formulated amount of 1,3-diolein-2-linolein; heating the weighed glyceride and the water phase separately to 60-70° C., then mixing them and emulsifying the mixture in a high pressure homogenizer, wherein the low pressure is 5-12 MPa and the high pressure is 25-50 MPa; repeating the cycle of homogenization for 3-6 times until the amount of particles below 2 μm is no less than 95% and particles above 5 μm are undetectable; if necessary, using NaOH or HCl to adjust pH to 4.8 to 8.5, preferably 6.8 to 7.0, most preferably 6.8; and

filtering the resulting homogeneous emulsion by nitrogen pressure through a ≦3 μm microporus filter; filling the emulsion with nitrogen, sterilizing and cooling the emulsion to afford the injection of the invention.

Preferably, the pharmaceutical preparation of the invention is a capsule comprising the following components:

| | | |
|---|---|---|
| 1,3-diolein-2-linolein | 200-800 | g |
| Antioxidant(s) and/or emulsifier(s) | 0.20-0.60 | g |
| To give | 1000 | capsules. |

The above capsule is prepared by a method comprising steps of:

preparing a glue solution: weighing gelatin, purified water, glycerin and a preservative at a weight ratio of 1:0.6-1.2:0.3-0.8:0.0001-0.01; adding glycerin, purified water and preservative sequentially into a glue melting tank; heating to 70-90; then adding gelatin and constantly stirring the mixture under vacuum until the gelatin is completely dissolved; filtering the glue solution and storing the filtered glue solution at 56-62° C. for use;

preparing a drug liquid: adding formulated amount of 1,3-diolein-2-linolein, antioxidant(s) and/or emulsifier(s) into an dosing tank, and stirring the mixture constantly until being homogeneously mixed; and

pressing capsules: choosing proper pellet dies according to the capsule size; pressing capsules at a temperature of 15-30 and a relative humidity of less than 35%; drying the pressed and shaped capsules; after removing capsules of abnormal size, washing the normal capsules with 95% medicinal ethanol, and drying them continuously to a moisture content of less than 12%; visually inspecting and removing unqualified capsules; finally printing and packaging to afford capsules.

In the above capsule, the preservative is selected from any one of 10% ethylparaben solution, benzoic acid, potassium sorbate and chlorhexidine acetate; the antioxidant is vitamin E; and the emulsifier is Tween 80.

It is shown in pharmacological experiments that the compound, 1,3-diolein-2-linolein, and pharmaceutical preparations thereof inhibit 7 human tumor cell lines in varying degrees, thus can be used as therapeutical drugs of neoplastic diseases.

Therefore, another aspect of the invention is to provide a use of the compound of formula (I) and pharmaceutical preparations thereof in the treatment of a tumor selected from a group consisting of pancreatic cancer, breast cancer, ovarian cancer, liver cancer, lung cancer, gastric cancer and colon cancer.

The following experimental data are used to illustrate beneficial anti-tumor effects of the compound of formula (I) and the pharmaceutical preparations thereof.

### I. Inhibition of the Compound of Formula (I) and Preparations Thereof on 7 Human Tumor Cell Lines in MTT Method In Vitro

A. Experimental Materials and the Preparation Thereof:
(1) Cell lines: PANC-1 (human pancreatic cancer cells), SKOV3 (human ovarian cancer cells), MCF-7 (human breast cancer cells), SMMC-7721 (human liver cancer cells), A549 (human lung cancer cells), BGC-823 (human gastric cancer cells), COLO205 (human colonic cancer cells), storaged and passaged maintainably in Research and Evaluation Center for Pharmacology, Shanghai Institute of Pharmaceutical Industry;
(2) DMEM complete medium supplied with 10% newborn calf serum (GIBCO BRL), 1% of penicillin (100 U/mL)+streptomycin (100 μg/mL);
(3) 0.25% trypsin solution, purchased from Invitrogen Corp. and storaged at −20;
(4) Phosphate buffer (PBS): NaCl 8 g, KCl 0.2 g, Na₂HPO₄ 1.15 g and KH₂PO₄ 0.2 g, dissolved in 1 L double-distilled water and autoclaved at 121 for 20 min, then storaged at 4;
(5) MTT (AMRESCO) solution: 5 mg/ml in PBS;
(6) Formazan crystal dissolving solution: SDS 10 g, isobutanol 5 ml and concentrated hydrochloric acid 0.1 ml, dissolved in 100 ml of deionized double distilled water.

B. Experimental Method

The inhibition effects of the compound of formula (I) on the above-mentioned cell lines were detected by using MTT method. The specific procedures were as follows:

(1) Cell culture: (a) Storaged cells were taken out from the liquid nitrogen, thawed quickly in a 37 water bath, then aseptically transferred into 6 ml of cellular medium in a 10 ml centrifugal tube, centrifuged at 1000 rpm for 5 min. The supernatant was discarded, then the precipitated cells were re-suspended in 5-6 ml cellular media by pipetting and transferred into a flask in a 37 incubator for cell culture; (b) Next day, the flask was taken out from the incubator and the used medium was discarded, then the cells were incubated in 5-6 ml fresh medium in the 37 incubator; (c) On the third day, the flask was taken out from the incubator and the used medium was discarded, then 2-3 ml of PBS (pH7.4) was added into the flask with rocking for cleaning it and the used PBS was discarded. Such a cellular cleaning step was repeated once again. 3-5 drops of 0.25% trypsin solution were added into the flask with sloshing, thus well-distributed in it. The flask was capped and placed in a 37 incubator for about 3 min, and the separation of cells from the flask wall was observed under the microscope. 2 ml of cellular medium was added and cells were separated completely from the flask wall by pipetting, then the cell suspension was transferred into 2 separate clean flasks, each containing 5-6 ml medium. The cell suspension was well-distributed by pipetting, then the flask was placed in a 37 incubator. (d) Step(c) was repeated every other day. In the whole cultivation process, adherent cells were not allowed to grow too dense and suspension cells were always maintained at a logarithmic growth stage.

(2) Preparation of the sample and the control: A proper amount of sample of the compound of formula (I) was dissolved in DMSO to obtain a solution in a concentration of 10 mg/ml. This solution was diluted in a gradient dilution with PBS to obtain a set of sample solutions in the concentration of 10 mg/ml, 5000 μg/ml, 2500 μg/ml, 1250 μg/ml, 625 μg/ml and 312.5 μg/ml, respectively.

(3) Each diluted sample solution was added into duplicated wells of a 96 well flat-bottom microplate (10 μl/well). The correspondingly diluted DMSO solutions, as controls, were added into the wells of the microplate.

(4) Cells in a logarithmic growth stage were trypsinized and washed, then re-suspended in the medium containing 10% calf serum. The number of living cells was counted in Trypan blue dye exclusion method and cell suspensions were adjusted into a density of 2×10⁵ cell/ml.

(5) The cell-contained 96 well flat-bottom microplate was placed in a 37 incubator and cells were incubated under 5% CO₂ for 48 h.

(6) 20 it of 5 mg/ml MTT solution was added into each well and cells were incubated continuously in the incubator for 3-4 h.

(7) 100 it of crystal dissolving solution was added into each well and cells were incubated continuously in the incubator overnight, so as to dissolve the resulted formazan crystals sufficiently. Then, the absorbance value was measured at 570 nm for each well.

(8) Based on absorbance values, inhibition rates on the cell growth were calculated for sample groups of various concentrations. The calculation formula was as follows:

(1−mean absorbance of experimental wells/mean absorbance of control wells)×100%

C. Experimental Results

TABLE 1
Inhibition rates of the compound of formula (I) in various
concentrations on the cell growth in 7 cell lines (%)

| | Concentration of the compound | | | | | |
|---|---|---|---|---|---|---|
| | 1000 | 500 | 250 | 125 | 62.5 | 31.25 |
| Cell line | μg/ml | μg/ml | μg/ml | μg/ml | μg/ml | μg/ml |
| PANC-1 | 75.00 | 48.15 | 25.89 | 2.01 | 0.80 | / |
| SKOV3 | 99.35 | 93.83 | 79.37 | 76.76 | 33.97 | 9.97 |
| MCF-7 | 99.73 | 68.80 | 67.09 | 62.26 | 31.48 | 13.30 |
| SMMC-7721 | 98.82 | 80.05 | 62.30 | 38.52 | 13.36 | 8.81 |
| A549 | 98.23 | 67.03 | 50.48 | 46.39 | 25.57 | 5.16 |
| BGC-823 | 98.52 | 84.96 | 76.15 | 78.96 | 63.83 | 8.31 |
| COLO205 | 93.77 | 82.30 | 43.93 | 12.62 | 10.66 | 8.95 |

TABLE 2
IC₅₀ values of the compound of formula (I) in 7 cell lines in vitro (μg/ml)

| | Cell line | | | | | | |
|---|---|---|---|---|---|---|---|
| Sample | PANC-1 | SKOV3 | MCF-7 | SMMC-7721 | A549 | BGC-823 | COLO205 |
| Positive control (Taxol) | 0.44 | 0.38 | 0.34 | 0.12 | 0.49 | 0.24 | 0.75 |
| compound of formula (I) | 508.9 | 92.61 | 105.7 | 150.2 | 155.6 | 84.98 | 209.3 |

D. Conclusion

It is shown in the results that the compound of formula (I) and preparations thereof in various concentrations have obvious inhibition on 7 human tumor cell lines. They can be used as antitumor drugs. Since the active ingredient in the pharmaceutical preparations has a simple construct and definite content, the clinical medication is safer. The less dose and controllable process quality of the compound and preparations thereof have avoided defects of more side effects resulting from the complex ingredients when Coix seed oil extracted from Coix seeds is administered directly.

### DESCRIPTION OF FIGURES

FIG. 1 is an EI mass spectrum of 1,3-diolein-2-linolein.

FIG. 2 is an APCI mass spectrum of 1,3-diolein-2-linolein.

FIG. 3 is an IR spectrum of 1,3-diolein-2-linolein.

FIG. 4 is a ¹H-NMR spectrum of 1,3-diolein-2-linolein.

FIG. 5 is a ¹³C-NMR spectrum of 1,3-diolein-2-linolein.

FIG. 6 is a HSQC spectrum of 1,3-diolein-2-linolein.

FIG. 7 is a HMBC spectrum of 1,3-diolein-2-linolein.

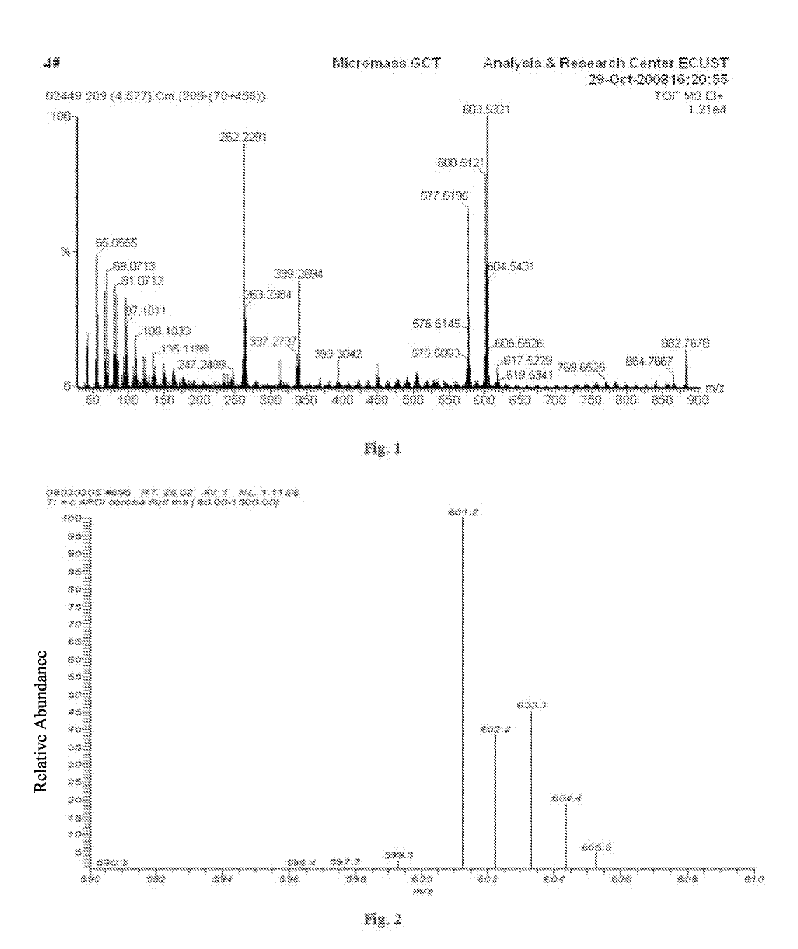

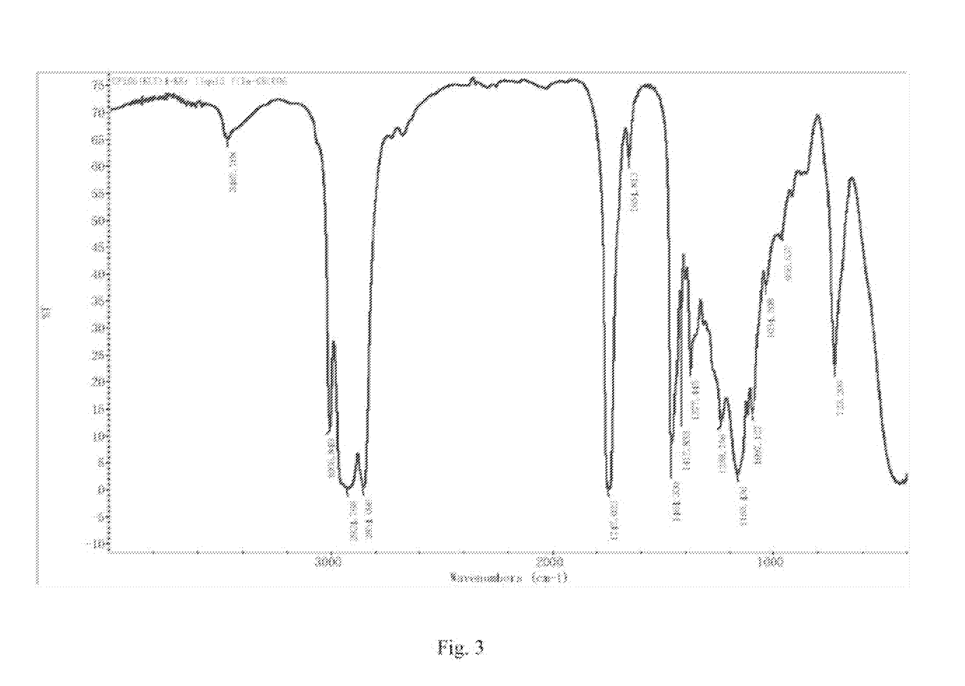

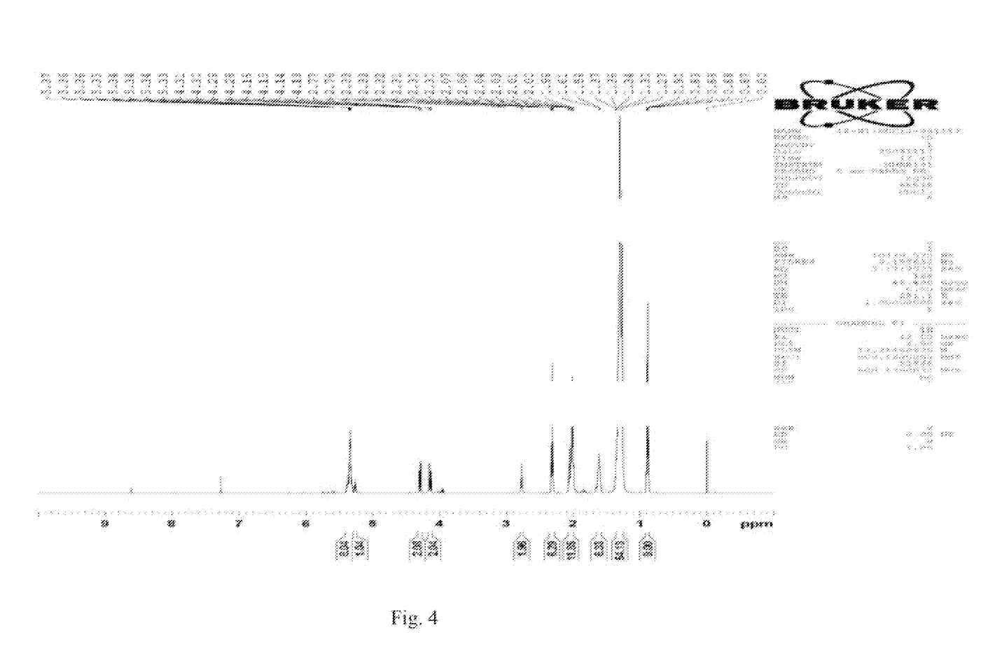

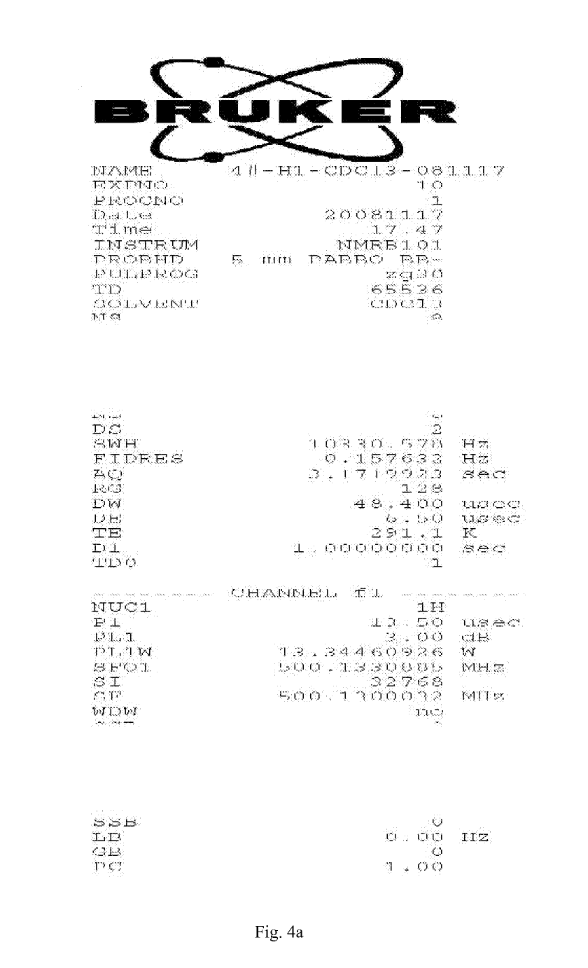

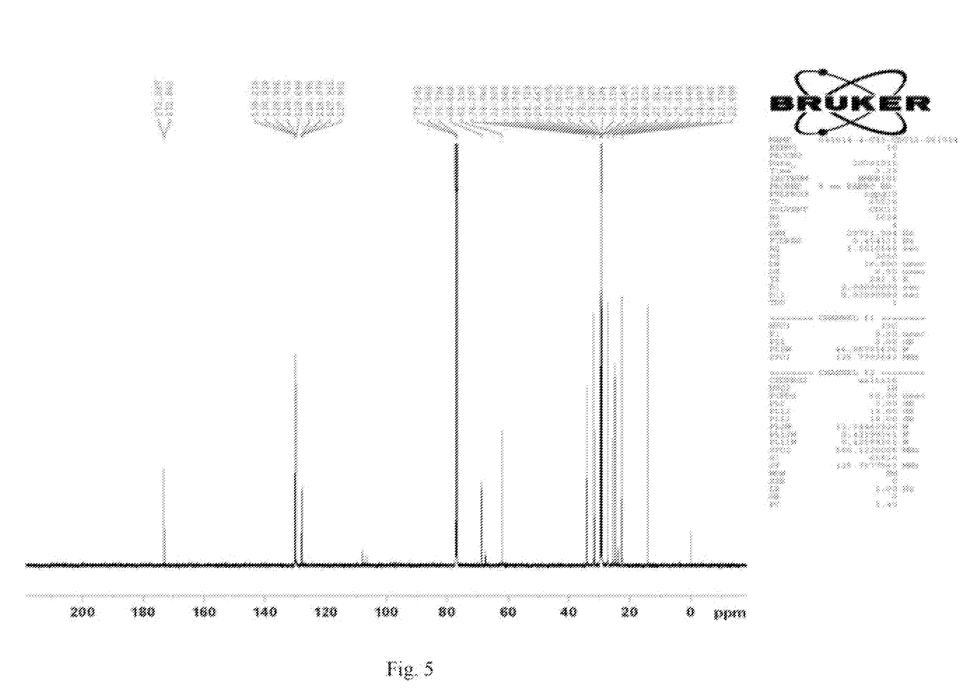

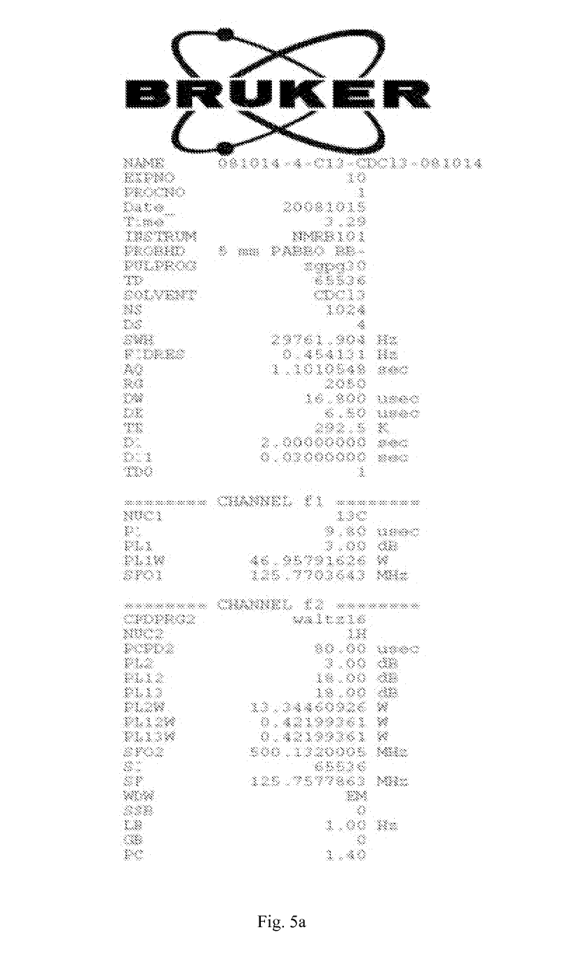

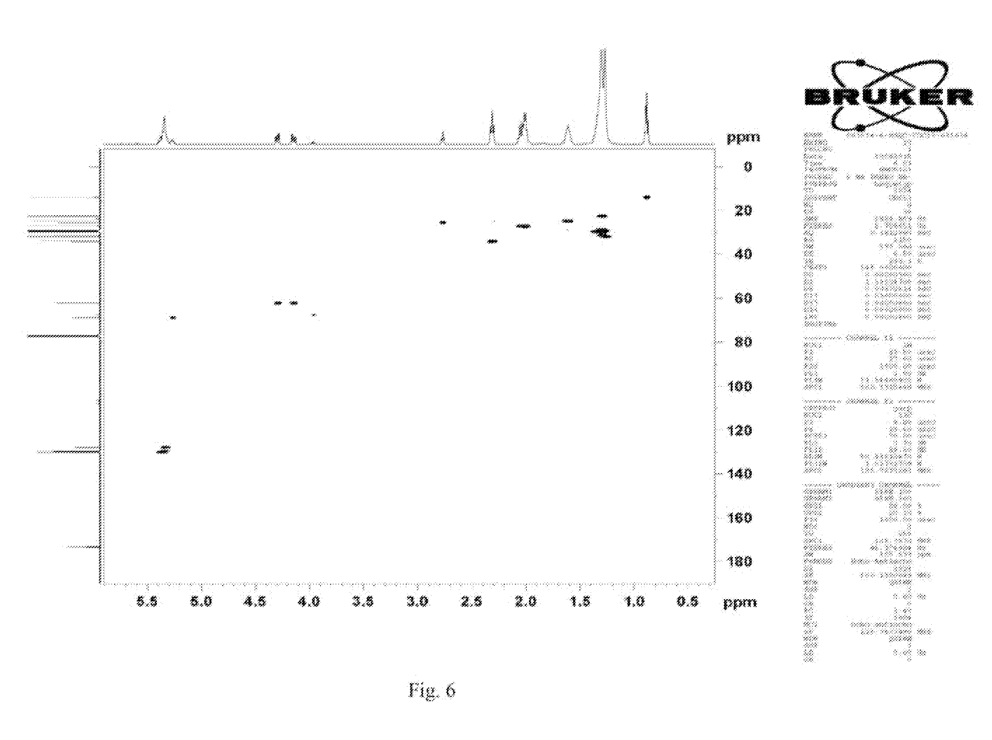

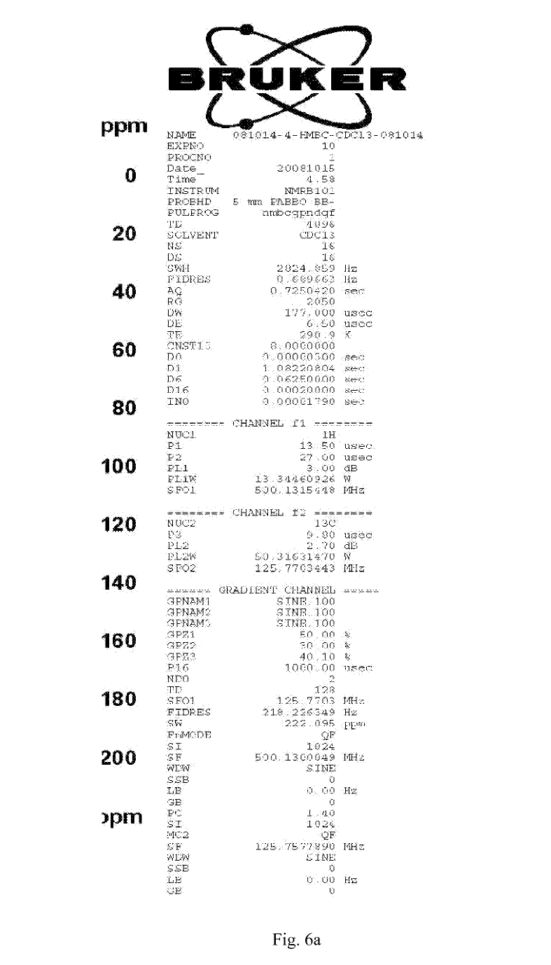

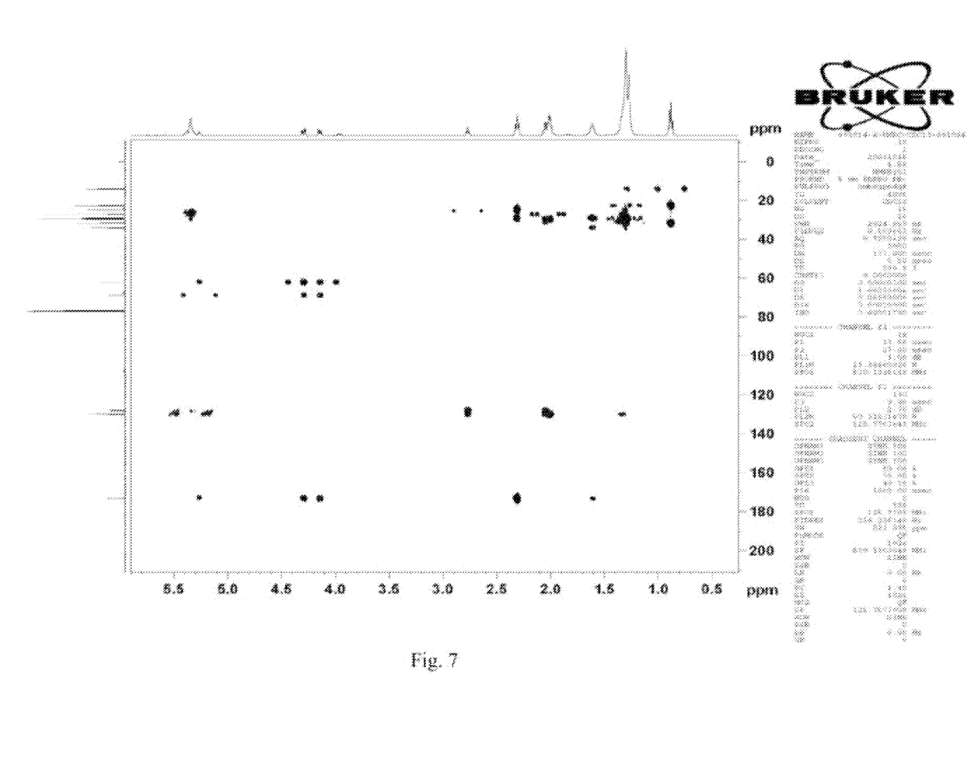

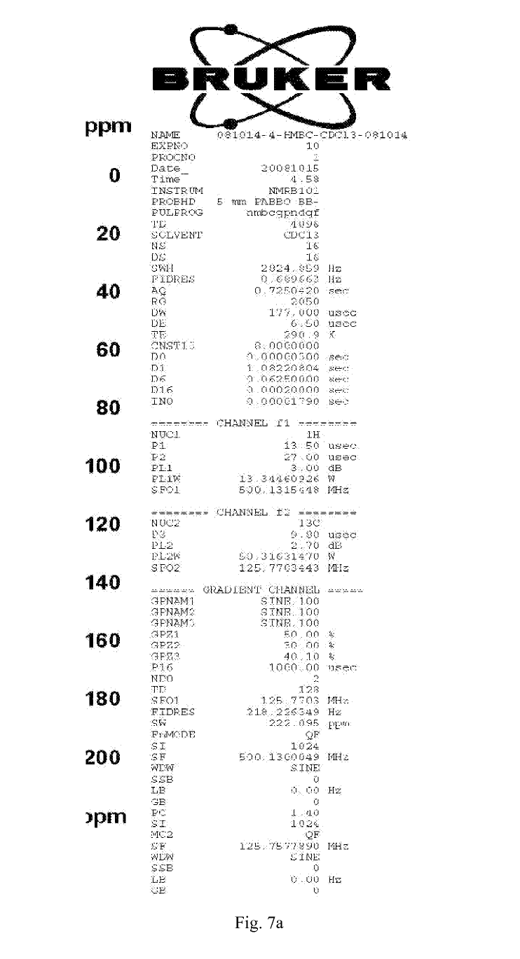

### DETAILED DESCRIPTION OF THE PREFERRED EMBODIMENTS

The following examples further illustrate the invention, but are not construed as a limitation of the invention.

### Example 1

### Preparation of Coix Seed Oil

1000 g Coix seeds having a moisture ≦10% were crushed into 10 mesh powder and extracted in a supercritical CO₂ extraction system, wherein the extraction temperature was 40° C., the extraction pressure was 20 Mpa, the separation temperature was 30, the separation pressure was 6 Mpa, and the flow rate of liquid CO₂ was 50 L/h. The Coix seed powder was continuously extracted for 2 h to give a crude Coix seed oil.

To the crude Coix seed oil obtained in the supercritical CO₂ extraction were added 40% petroleum ether by weight of the crude Coix seed oil and 0.5% NaOH aqueous solution to caustically refine the crude oil. After layering, to the organic phase was added 3% activated neutral alumina by weight of the crude oil, and the mixture was filtered. The filtrate was heated to 40, and 2% activated Kaolin by weight of the crude oil was added. The mixture was filtered and the filtrate was concentrated under a reduced pressure to remove the solvent. 8% activated neutral alumina by weight of the crude oil was added. The mixture was filtered, and the filtrated oil underwent dry heat sterilization by vacuum at 180. After cooling, the oil was filtered to give the Coix seed oil.

### Example 2

### Preparation of Coix Seed Oil

1000 g Coix seeds having a moisture ≦10% were crushed into 100 mesh powder and extracted in a supercritical CO₂ extraction system, wherein the extraction temperature was 50° C., the extraction pressure was 22 Mpa, the separation temperature was 40 and the separation pressure was 8 Mpa, and the flow rate of liquid CO₂ was 500 L/h. The Coix seed powder was continuously extracted for 3 h to give a crude Coix seed oil.

To the crude Coix seed oil obtained in the supercritical CO₂ extraction were added 46% petroleum ether by weight of the crude oil and 1% NaOH aqueous solution to caustically refine the crude oil. After layering, to the organic phase was added 5% activated neutral alumina by weight of the crude oil, and the mixture was filtered. The filtrate was heated to 50, then added 4% activated Kaolin by weight of the crude oil and the mixture was filtered. The filtrate was concentrated under a reduced pressure to remove the solvent. Then 10% activated neutral alumina by weight of the crude oil was added. The mixture was filtered, and the filtrated oil underwent dry heat sterilization by vacuum at 150. After cooling, the oil was filtered to give the Coix seed oil.

### Example 3

### Preparation of Coix Seed Oil

1000 g Coix seeds having a moisture ≦10% were crushed into 200 mesh powder and extracted in a supercritical CO₂ extraction system, wherein the extraction temperature was 60° C., the extraction pressure was 24 Mpa, the separation temperature was 50, the separation pressure was 10 Mpa, and the flow rate of liquid CO₂ was 1500 L/h. The Coix seed powder was continuously extracted for 1.5 h to give a crude Coix seed oil.

To the crude Coix seed oil obtained in the supercritical CO₂ extraction were added 50% petroleum ether by weight of the crude oil and 2% NaOH aqueous solution to caustically refine the crude oil. After layering, to the organic phase was added 6% activated neutral alumina by weight of the crude oil, and the mixture was filtered. The filtrate was heated to 45 and 5% activated Kaolin by weight of the crude oil was added. Then the mixture was filtered and the filtrate was concentrated under a reduced pressure to remove the solvent. 10% Activated neutral alumina by weight of the crude oil was added. The mixture was filtered and the filtrated oil underwent dry heat sterilization by vacuum at 160. After cooling, the oil was filtered to give the Coix seed oil.

### Example 4

### Preparation of Coix Seed Oil

1000 g Coix seeds having a moisture ≦10% were crushed into 100 mesh powder and extracted in a supercritical CO₂ extraction system. Coix seed powder was put in an extractor. The CO₂ preheater, extractor and separation column were heated, so that the extraction temperature was 60, the extraction pressure was 24 Mpa, the separation temperature was 60 and the separation pressure was 15 Mpa. The supercritical CO₂ gas with the extracted oil was introduced, as needed, into 5 resolution columns wherein the resolution temperature was 50 and the resolution pressure was 6 Mpa. The flow rate of CO₂ in the whole cycle was kept at 3000 L/h. The continuous extraction for 4 h afforded a crude Coix seed oil from the lower end of the separation column.

To the crude Coix seed oil obtained in the supercritical CO₂ extraction were added 58% petroleum ether by weight of the crude oil and 2% NaOH aqueous solution, q.s., to caustically refine the crude oil. After layering, the organic phase was washed with purified water and the washed organic phase was demulsified with acetone, q.s. To the upper oil layer was added 8% activated neutral alumina by weight of the crude oil, and the mixture was filtered. The filtrate was heated to 46 and 6% activated Kaolin by weight of the crude oil was added. Then the mixture was filtered, and the filtrate was concentrated under a reduced pressure to remove the solvent. 12% Activated neutral alumina by weight of the crude oil was added. The mixture was filtered and the filtrated oil underwent dry heat sterilization by vacuum at 170 for 2 h. After cooling, the oil was filtered through a 0.2 μm millipore filter to give the Coix seed oil.

### Example 5

### Preparation of Coix Seed Oil

1000 g Coix seeds having a moisture 10% were crushed into 300 mesh powder and extracted in a supercritical CO₂ extraction system. Coix seed powder was put in an extractor. The CO₂ preheater, extractor and separation column were heated, so that the extraction temperature was 50, the extraction pressure was 20 Mpa, the separation temperature was 45 and the separation pressure was 12 Mpa. The supercritical CO₂, post extraction of coix seed oil, was introduced, as needed, into 3 resolution columns wherein the resolution temperature was 45 and the resolution pressure was 5 Mpa. The flow rate of CO₂ in the whole cycle was kept at 2000 L/h. The continuous extraction for 3 h affords a crude Coix seed oil from the lower end of the separation column.

To the crude Coix seed oil obtained in the supercritical CO₂ extraction were added 48% petroleum ether by weight of the crude oil and 2% NaOH aqueous solution, q.s., to caustically refine the crude oil. After layering, the organic phase was washed with purified water and the washed organic phase was demulsified with acetone, q.s. To the upper oil layer was added 6% activated neutral alumina by weight of the crude oil, and the mixture was filtered. The filtrate was heated to 42 and 4% activated Kaolin by weight of the crude oil was added. Then the mixture was filtered, and the filtrate was concentrated under a reduced pressure to remove the solvent. 9% Activated neutral alumina by weight of the crude oil was added. The mixture was filtered and the filtrated oil underwent dry heat sterilization by vacuum at 165 for 3 h. After cooling, the oil was filtered through a ≦0.2 μm millipore filter to give the Coix seed oil.

### Example 6

### Preliminary Separation of 1,3-Diolein-2-Linolein

Instrument and method: A preparative Gilson high performance liquid chromatograph was used in combination with an evaporative light-scattering detector, wherein the preparative chromatographic column was Agilent Zorbax SB-C18 (250 mm×21.2 mm, 7 μm); mobile phase A was acetonitrile and mobile phase B was tetrahydrofuran/acetonitrile (1:1); gradient conditions were mobile phase B: 0-27 min: 50%-60%, 27-35 min: 60-90%, 35-45 min: 90-100%; the flow rate was 18 mL/min; the injection volume was 200-300 μl; and the collection response was set at the peak level min=30 mV.

Preparation of samples: 0.1 g/ml Coix seed oil solution was prepared with mobile phase B.

Collection of fractions: The fraction of the chromatographic peak at retention time of about 16.0 min was collected and the solvent was removed at a low temperature and a reducing pressure to obtain a crude 1,3-diolein-2-linolein.

### Example 7

### Double-Separation of 1,3-diolein-2-linolein

First separation: The crude 1,3-diolein-2-linolein obtained in Example 6 was dissolved in n-hexane, q.s., with an ultrasonic wave, to obtain a solution; a sample (60 μl) of this solution was analyzed in an analytical Agilent 1100 high performance liquid chromatograph (column: Venusil XBP C18 (L) (10*250 mm, 5 μm, 150 Å)) with a mobile phase, acetonitrile/tetrahydrofuran (75:25), at a flow rate of 4 mL/min. Fractions were detected with a DAD detector at 205 nm. The fraction of the chromatographic peak at the retention time of about 19 min was collected and concentrated with nitrogen flow blowing.

Second separation: The product obtained in the first separation was dissolved in n-hexane, q.s., with an ultrasonic wave, to obtain a solution. A sample (60 μl) of this solution was injected in a high performance liquid chromatograph (column: Venusil CA (10*250 mm, 5 μm, 1000 Å)) and enluted with a mobile phase, n-hexane/ethanol (95:5), at a flow rate of 4 mL/min. Fractions were detected with an ultraviolet detector at 205 nm and the chromatographic peak at retention time of about 2.5-5 min was collected. The fraction was concentrated with nitrogen flow blowing, and the final product, 1,3-diolein-2-linolein, was cryopreserved at a low temperature.

### Example 8

### Preparation of the Injection of 1,3-diolein-2-linolein

Formulation:

| | | |
|---|---|---|
| 1,3-diolein-2-linolein | 200 | g |
| Soybean lecithin for injection | 25 | g |
| Glycerin acceptable for injection | 30 | g |
| Water for injection adds to | 1000 | mL |

Process:

To a formulated amount of soybean lecithin for injection was added an appropriate amount of water for injection. The mixture was dispersed with a high shear dispersing emulsifier into a dispersion without bulk or granular solid. Formulated amount of glycerin acceptable for injection was added. Then water for injection was added to a specified amount, and the mixture was stirred to give a water phase.

A formulated amount of 1,3-diolein-2-linolein was weighed. The weighed oil and the water phase prepared above were heated separately to 65, then mixed and emulsified in a high pressure homogenizer, wherein the low pressure was 8 MPa and the high pressure was 30 MPa. The homogenization was repeated for 4 times until the amount of particles below 2 μm was no less than 95% and particles above 5 μm were undetectable. If necessary, NaOH or HCl was used to adjust the pH to 6.8.

The resulting homogeneous emulsion was filtered by nitrogen pressure through a ≦3 μm microporus filter, then filled under nitrogen, and finally sterilized and cooled to afford the injection.

### Example 9

### Preparation of the Capsule of 1,3-diolein-2-linolein

Formulation:

| | | |
|---|---|---|
| 1,3-diolein-2-linolein | 500 | g |
| Vitamin E | 0.40 | g |
| to give | 1000 | capsules |

Process:

Glue formulation: Gelatin, purified water, glycerin and potassium sorbate solution were weighed at a weight ratio of 1:0.9:0.6:0.005. Glycerin, purified water and potassium sorbate solution were sequentially added into a glue melting tank and the mixture was heated to 80. Then gelatin was added and the mixture was constantly stirred under vacuum until the gelatin was completely dissolved. The glue was filtered and stored at 56-62 for use.

Drug liquid formulation: Formulated amount of 1,3-diolein-2-linolein and Vitamin E were added into an ingredient tank and the mixture was stirred constantly until being thoroughly mixed.

Capsule pressing: Proper pellet dies were chosen according to the capsule size. Capsules were pressed at a temperature of 15 and a relative humidity of less than 35%, then shaped and dried. After excluding capsules of abnormal size, normal capsules were washed with 95% medicinal ethanol and dried continuously till the moisture content was less than 12%. Unqualified capsules were removed by visual inspection, and final products were printed and packaged.

### Example 10

### Preparation of the Injection of 1,3-diolein-2-linolein

Formulation:

| | | |
|---|---|---|
| 1,3-diolein-2-linolein | 100 | g |
| Soybean lecithin for injection | 10 | g |
| Glycerin for injection | 15 | g |
| Water for injection adds to | 1000 | mL |

Process:

To a formulated amount of soybean lecithin for injection was added an appropriate amount of water for injection. The mixture was dispersed with a high shear dispersing emulsifier into a dispersion without bulk or granular solid. Formulated amount of glycerin for injection was added. Then water for injection was added to a specified amount, and the mixture was stirred to give a water phase.

A formulated amount of 1,3-diolein-2-linolein was weighed. The weighed oil and the water phase prepared above were heated separately to 60, then mixed and emulsified in a high pressure homogenizer, wherein the low pressure was 5 MPa and the high pressure was 35 MPa. The homogenization was repeated for 6 times until the amount of particles below 2 μm was no less than 95% and particles above 5 μm were undetectable. If necessary, NaOH or HCl was used to adjust the pH to 4.8.

The resulting homogeneous emulsion was filtered by nitrogen pressure through a ≦3 μm microporus filter, then filled under nitrogen, and finally sterilized and cooled to afford the injection.

### Example 11

### Preparation of the Injection of 1,3-diolein-2-linolein

Formulation:

| | | |
|---|---|---|
| 1,3-diolein-2-linolein | 300 | g |
| Soybean lecithin acceptable for injection | 40 | g |
| Glycerin acceptable for injection | 50 | g |
| Water for injection adds to | 1000 | mL |

Process:

To a formulated amount of soybean lecithin acceptable for injection was added an appropriate amount of water for injection. The mixture was dispersed with a high shear dispersing emulsifier into a dispersion without bulk or granular solid. Formulated amount of glycerin acceptable for injection was added. Then water for injection was added to a specified amount, and the mixture was stirred to give a water phase.

A formulated amount of 1,3-diolein-2-linolein was weighed. The weighed oil and the water phase prepared above were heated separately to 70, then mixed and emulsified in a high pressure homogenizer, wherein the low pressure was 11 MPa and the high pressure was 45 MPa. The homogenization was repeated for 3 times until the amount of particles below 2 μm was no less than 95% and particles above 5 μm were undetectable. If necessary, NaOH or HCl was used to adjust the pH to 8.5.

The resulting homogeneous emulsion was filtered by nitrogen pressure through a ≦3 μm microporus filter, then filled under nitrogen, and finally sterilized and cooled to afford the injection.

### Example 12

### Preparation of the Injection of 1,3-diolein-2-linolein

Formulation:

| | | |
|---|---|---|
| 1,3-diolein-2-linolein | 150 | g |
| Soybean lecithin acceptable for injection | 35 | g |
| Glycerin for injection | 30 | g |
| Water for injection adds to | 1000 | mL |

Process:

To a formulated amount of soybean lecithin acceptable for injection was added an appropriate amount of water for injection. The mixture was dispersed with a high shear dispersing emulsifier into a dispersion without bulk or granular solid. Formulated amount of glycerin for injection was added. Then water for injection was added to a specified amount, and the mixture was stirred to give a water phase.

A formulated amount of 1,3-diolein-2-linolein was weighed. The weighed oil and the water phase prepared above were heated separately to 68, then mixed and emulsified in a high pressure homogenizer, wherein the low pressure was 9 MPa and the high pressure was 40 MPa. The homogenization was repeated for 5 times until the amount of particles below 2 μm was no less than 95% and particles above 5 μm were undetectable. If necessary, NaOH or HCl was used to adjust the pH to 7.2.

The resulting homogeneous emulsion was filtered by nitrogen pressure through a ≦3 μm microporus filter, then filled under nitrogen, and finally sterilized and cooled to afford the injection.

### Example 13

### Preparation of the Capsule of 1,3-diolein-2-linolein

Formulation:

| | | |
|---|---|---|
| 1,3-diolein-2-linolein | 200 | g |
| Vitamin E | 0.20 | g |
| to give | 1000 | capsules |

Process:

Glue formulation: Gelatin, purified water, glycerin and 10% ethylparaben solution were weighed at a weight ratio of 1:1.2:0.8:0.01. Glycerin, purified water and 10% ethylparaben solution were sequentially added into a glue melting tank and heated to 70. Then gelatin was added and the mixture was constantly stirred under vacuum until the gelatin was completely dissolved. The glue was filtered and stored at 60 for use.

Drug liquid formulation: Formulated amount of 1,3-diolein-2-linolein and Vitamin E were added into an ingredient tank and the mixture was stirred constantly until being thoroughly mixed.

Capsule pressing: Proper pellet dies were chosen according to the capsule size. Capsules were pressed at a temperature of 30 and a relative humidity of less than 35%, then shaped and dried. After excluding capsules of abnormal size, normal capsules were washed with 95% medicinal ethanol and dried continuously till the moisture content was less than 12%. Unqualified capsules were removed by visual inspection, and final products were printed and packaged.

### Example 14

### Preparation of the Capsule of 1,3-diolein-2-linolein

Formulation:

| | | |
|---|---|---|
| 1,3-diolein-2-linolein | 800 | g |
| Tween 80 | 0.60 | g |
| to give | 1000 | capsules |

Process:

Glue formulation: Gelatin, purified water, glycerin and benzoic acid were weighed at a weight ratio of 1:1.2:0.8:0.01. Glycerin, purified water and benzoic acid were sequentially added into a glue melting tank and heated to 90. Then gelatin was added and constantly stirred under vacuum until the gelatin was completely dissolved. The glue was filtered and stored at 56 for use.

Drug liquid formulation: Formulated amount of 1,3-diolein-2-linolein and Tween 80 were added into an ingredient tank and the mixture was stirred constantly until being thoroughly mixed.

Capsule pressing: Proper pellet dies were chosen according to the capsule size. Capsules were pressed at a temperature of 25 and a relative humidity of less than 35%, then shaped and dried. After excluding capsules of abnormal size, normal capsules were washed with 95% medicinal ethanol and dried continuously till the moisture content was less than 12%. Unqualified capsules were removed by visual inspection, and final products were printed and packaged.

### Example 15

### Preparation of the Capsule of 1,3-diolein-2-linolein

Formulation:

| | | |
|---|---|---|
| 1,3-diolein-2-linolein | 600 | g |
| Tween 80 | 0.30 | g |
| to give | 1000 | capsules |

Process:

Glue formulation: Gelatin, purified water, glycerin and chlorhexidine acetate were weighed at a weight ratio of 1:1.0:0.5:0.008. Glycerin, purified water and chlorhexidine acetate were sequentially added into a glue melting tank and heated to 85. Then gelatin was added and the mixture was constantly stirred under vacuum until the gelatin was completely dissolved. The glue was filtered and stored at 62 for use.

Drug liquid formulation: Formulated amount of 1,3-diolein-2-linolein and Tween 80 were added into an ingredient tank and the mixture was stirred constantly until being thoroughly mixed.

Capsule pressing: Proper pellet dies were chosen according to the capsule size. Capsules were pressed at a temperature of 28 and a relative humidity of less than 35%, then shaped and dried. After excluding capsules of abnormal size, normal capsules were washed with 95% medicinal ethanol and dried continuously till the moisture content was less than 12%. Unqualified capsules were removed by visual inspection, and final products were printed and packaged.

What is claimed is:

1. A compound, 1,3-diolein-2-linolein, having a structure of formula (I):

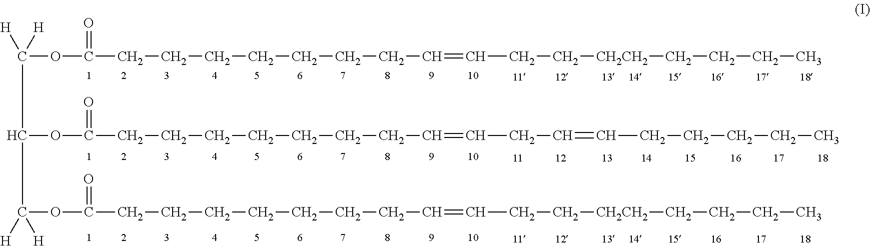

2. The compound of claim 1, which is prepared by the process comprising steps of:
(1) preparation of Coix seed oil;
(2) preliminary separation of 1,3-diolein-2-linolein; and
(3) double-separation of 1,3-diolein-2-linolein.

3. The compound of claim 2, wherein said step (1) comprises steps of:
crushing Coix seeds having a moisture content ≦10% into 10-300 mesh powder and extracting said powder using a supercritical fluid extraction to afford a crude Coix seed oil; adding petroleum ether in an amount of 40-58% by weight of crude Coix seed oil and caustically refining said crude Coix seed oil with aqueous alkali, q.s.; after layering, adding neutral alumina and/or kaolin to the organic phase; filtering and sterilizing the filtrated oil via dry heat sterilization under vacuum at 150-180° C.; then cooling and filtering the sterilized oil to give Coix seed oil.

4. The compound of claim 3, wherein:
said supercritical fluid extraction is the supercritical CO₂ extraction with an extraction temperature of 30-60° C., an extraction pressure of 19-24 Mpa, a separation temperature of 30-60° C., a separation pressure of 6-15 Mpa, a carbon dioxide flow rate of 10-5000 L/h, and an extraction time of 1-4 h;
said aqueous alkali is a 0.5-3% KOH or NaOH aqueous solution; and
said step of adding neutral alumina and/or kaolin includes adding 3-8% activated neutral alumina by weight of crude Coix seed oil; filtering the mixture and heating the filtrate to 40-50° C.; adding 2-6% activated Kaolin by weight of crude Coix seed oil and filtering the mixture; removing solvent from the filtrate under vacuum; and adding 8-12% activated neutral alumina by weight of crude Coix seed oil, then filtering the mixture.

5. The compound of claim 2, wherein step (1) especially comprises steps of:
crushing Coix seeds having a moisture content ≦10% into 10-300 mesh powder and extracting said powder in a supercritical CO₂ extraction system wherein the CO₂ preheater, extractor and separation column are heated to reach the extraction temperature of 30-60° C. and separation temperature of 30-60° C., and pressurized to reach the extraction pressure of 19-24 Mpa and separation pressure of 6-15 Mpa; the CO₂, post the extraction of coix seed oil, enters into one or more resolution column(s), as needed, having a resolution temperature of 30-60 and a resolution pressure of 2-6 Mpa. The flow rate of CO₂ in the whole cycle is kept at 10-5000 L/h. The continuous extraction for 1-4 h affords a crude Coix seed oil from the lower end of the separation column; and
adding petroleum ether in an amount of 40-58% by weight of crude Coix seed oil to the crude Coix seed oil, and caustically refining the mixture with 2% NaOH solution, q.s.; after layering, collecting the organic layer and washing it with purified water; demulsifying the washed organic layer with acetone (q.s.); to the upper layer oil, adding activated neutral alumina in an amount of 3-8% by weight of crude Coix seed oil, and filtering the mixture; heating the filtrate up to 40-50 and adding activated kaolin in an amount of 2-6% by weight of crude Coix seed oil, and filtering the mixture; removing solvent from the filtrate under vacuum; and adding activated neutral alumina in an amount of 8-12% by weight of crude Coix seed oil, then filtering the mixture to give an oil; sterilizing the filtrated oil for 1-3 h via dry heat sterilization under vacuum at 150-180° C.; then cooling and filtering the sterilized oil through a ≦0.2 μm microporus membrane to give Coix seed oil

6. The compound of claim 2, wherein said preliminary separation of 1,3-diolein-2-linolein in step (2) is especially as follows:
Instrument and method: using a preparative high performance liquid chromatograph in combination with an evaporative light-scattering detector, wherein the preparative chromatographic column is Agilent Zorbax SB-C18 (250 mm×21.2 mm, 7 μm); mobile phase A is acetonitrile and mobile phase B is tetrahydrofuran/acetonitrile (1:1); gradient conditions are mobile phase B: 0-27 min: 50%-60%, 27-35 min: 60-90%, 35-45 min: 90-100%; the flow rate is 18 mL/min; injection volume is 200-300 μl; and the collection response is set at the peak level min=30 mV;
Preparation of sample: 0.1 g/ml Coix seed oil solution, prepared with mobile phase B; and
Collection of fraction: collecting the fraction of the chromatographic peak at the retention time of about 16 min and removing the solvent at a low temperature and a reducing pressure to obtain a preliminarily prepared sample.

7. The compound of claim 2, wherein said double-separation of 1,3-diolein-2-linolein in step (3) is especially as follows:
First separation: dissolving the sample obtained in said preliminary separation in n-hexane, q.s., with ultrasonic wave to obtain a solution; injecting a sample (60 μl) of said solution in an analytical high performance liquid chromatograph (column: Venusil XBP C18 (L) (10*250 mm, 5 μm, 150 Å)); eluting the sample with a mobile phase: acetonitrile/tetrahydrofuran (75:25) at a flow rate of 4 mL/min; detecting fractions with a DAD detector at 205 nm; collecting the fraction of chromatographic peak at the retention time of about 19 min and concentrating said fraction with nitrogen flow blowing; and
Second separation: dissolving the sample obtained in the first separation in n-hexane, q.s., with an ultrasonic wave, to obtain a solution; injecting a sample (60 μl) of said solution in a high performance liquid chromatograph (column: Venusil CA (10*250 mm, 5 μm, 1000 Å)); eluting the sample with a mobile phase: n-hexane/ethanol (95:5) at a flow rate of 4 mL/min; detecting fractions with an ultraviolet detector, at 205 nm; collecting the fraction of the chromatographic peak at retention time of about 2.5-5 min and concentrating said fraction with a nitrogen flow; and cryopreserved the final product at a low temperature.

8. A pharmaceutical preparation, comprising a therapeutically effective amount of the compound of claim 1 and one or more pharmaceutically acceptable carriers.

9. The pharmaceutical preparation of claim 8, wherein said pharmaceutically acceptable carriers are selected from pharmaceutically conventional dilutions, excipients, fillers, emulsifiers, binders, lubricants, absorption accelerators, surfactants, disintegrants, lubricants and stabilizers, if necessary, flavoring agents, sweeteners, preservative and/or coloring agents.

10. The pharmaceutical preparation of claim 9, wherein said pharmaceutically acceptable carriers are selected from one or more in the group consisting of: mannitol, sorbitol, sodium metabisulfite, sodium bisulfite, sodium thiosulfate, cysteine hydrochloride, thioglycolic acid, methionine, soybean lecithin, vitamin C, vitamin E, EDTA disodium, EDTA calcium sodium, a monovalent alkali metal carbonate, acetate, phosphate or its aqueous solution, hydrochloric acid, acetic acid, sulfuric acid, phosphoric acid, amino acids, sodium chloride, potassium chloride, sodium lacetate, ethylparaben solution, benzoic acid, potassium sorbate, chlorhexidine acetate, xylitol, maltose, glucose, fructose, dextran, glycine, starch, sucrose, lactose, mannitol, and silicic derivatives, cellulose and its derivatives, alginates, gelatin, polyvinyl pyrrolidone, glycerin, Tween 80, agar-agar, calcium carbonate, calcium bicarbonate, surfactant, polyethylene glycol, cyclodextrin, β-cyclodextrin, phospholipid material, kaolin, talc, and calcium stearate or magnesium stearate.

11. The pharmaceutical preparation of claim 8, wherein said pharmaceutical preparation is an oral solid preparation, an oral liquid preparation, or an injection.

12. The pharmaceutical preparation of claim 11, wherein:
said oral solid preparation is selected from any one of capsules, tablets, dripping pills, granules and concentrated pills;
said oral liquid preparation is selected from any one of aqueous or oily suspensions, solutions, emulsions, syrups or elixirs, and a dry product that can be reconstructed by water or other suitable carrier before use; and
said injection is selected from any one of injection, lyophilized powder and aqueous injection.

13. The pharmaceutical preparation of claim 12, wherein said injection comprises the following components:

| | | |
|---|---|---|
| 1,3-diolein-2-linolein | 100-300 | g |
| Soybean lecithin for Injection or | 10-40 | g |
| soybean lecithin acceptable for injection | | |
| Glycerin for Injection or | 15-50 | g |
| glycerin acceptable for injection | | |
| Water for injection adds to | 1000 | mL. |

14. The pharmaceutical preparation of claim 13, which is prepared by a method comprising steps of:
adding appropriate amount of water for injection to a formulated amount of soybean lecithin for injection or soybean lecithin acceptable for injection; dispersing the mixture with a high shear dispersing emulsifier to give a dispersion without bulk or granular solid; adding a formulated amount of glycerin for injection or glycerin acceptable for injection; then adding water for injection to a specified amount, and stirring the mixture to give a water phase;
weighing a formulated amount of 1,3-diolein-2-linolein; heating the weighed glyceride and the water phase separately to 60-70° C., then mixing them and emulsifying the mixture in a high pressure homogenizer, wherein the low pressure is 5-12 MPa and the high pressure is 25-50 MPa; repeating the cycle of homogenization for 3-6 times until the amount of particles below 2 μm is no less than 95% and particles above 5 μm are undetectable; if necessary, using NaOH or HCl to adjust the pH to 4.8 to 8.5, preferably 6.8 to 7.0, most preferably 6.8; and
filtering the resulting homogeneous emulsion by nitrogen pressure through a ≦3 μm microporus filter; filling the emulsion in nitrogen, sterilizing and cooling the emulsion to afford the injection.

15. The pharmaceutical preparation of claim 12, wherein said capsule comprises the following components:

| | | |
|---|---|---|
| 1,3-diolein-2-linolein | 200-800 | g |
| Antioxidant(s) and/or emulsifier(s) | 0.20-0.60 | g |
| To give | 1000 | capsules. |

16. The pharmaceutical preparation of claim 15, which is prepared by a method comprising steps of:
preparing glue solution: weighing gelatin, purified water, glycerin and a preservative at a weight ratio of 1:0.6-1.2:0.3-0.8:0.0001-0.01; adding glycerin, purified water and preservative sequentially into a glue melting tank; heating to 70-90; then adding gelatin and constantly stirring the mixture under vacuum until the gelatin is completely dissolved; filtering the glue solution and storing the filtered glue solution at 56-62° C. for use;
preparing drug liquid: adding formulated amount of 1,3-diolein-2-linolein, antioxidant(s) and/or emulsifier(s) into an dosing tank, and stirring the mixture constantly until being homogeneously mixed; and
pressing capsules: choosing proper pellet dies according to the capsule size; pressing capsules in a temperature of 15-30 and a relative humidity of less than 35%; drying the pressed and shaped capsules; after removing capsules of abnormal size, washing the normal capsules with 95% medicinal ethanol, and drying them continuously to a moisture content of less than 12%; visually inspecting and removing unqualified capsules; finally printing and packaging to afford capsules.

17. The pharmaceutical preparation of claim 16, wherein:
said preservative is selected from any one of 10% ethylparaben solution, benzoic acid, potassium sorbate and chlorhexidine acetate;
said antioxidant is vitamin E; and
said emulsifier is Tween 80.

18. Use of the compound of claim 1 in the treatment of a tumor selected from a group consisting of pancreatic cancer, breast cancer, ovarian cancer, liver cancer, lung cancer, gastric cancer and colon cancer.

19. Use of the pharmaceutical preparation of claim 8 in the treatment of a tumor selected from a group consisting of pancreatic cancer, breast cancer, ovarian cancer, liver cancer, lung cancer, gastric cancer and colon cancer.
